(19) United States
(12) Patent Application Publication
(10) Pub. No.: US 2021/0321962 A1
(43) Pub. Date: Oct. 21, 2021

(54) IMAGE SENSOR HAVING RADIATION DETECTORS OF DIFFERENT ORIENTATIONS

(71) Applicant: SHENZHEN XPECTVISION TECHNOLOGY CO., LTD., Shenzhen (CN)

(72) Inventors: Wufeng WANG, Shenzhen (CN); Xiaoming LAN, Shenzhen (CN); Chongshen SONG, Shenzhen (CN); Zhaojian YU, Shenzhen (CN)

(21) Appl. No.: 17/365,359

(22) Filed: Jul. 1, 2021

Related U.S. Application Data
(63) Continuation of application No. PCT/CN2019/071119, filed on Jan. 10, 2019.

(51) Int. Cl. A61B 6/00; A61B 6/03; G01T 1/24

(52) U.S. Cl. CPC A61B 6/4208; A61B 6/502; G01T 1/24; A61B 6/4429; A61B 6/032

(57) ABSTRACT

Disclosed herein is an image sensor comprising: a first, a second, and a third radiation detectors, each of which comprising a planar surface to receive radiation from a radiation source; wherein the planar surfaces of the first radiation detector and the second radiation detector are not parallel, the planar surfaces of the second radiation detector and the third radiation detector are not parallel, and the planar surfaces of the third radiation detector and the first radiation detector are not parallel; wherein the first radiation detector, the second radiation detector and the third radiation detector are not arranged in the same row; wherein the first, the second and the third radiation detectors are configured such that the planar surface of each of them includes a position at which an angle of incidence of the radiation from the radiation source is 0°.

### BACKGROUND

Radiation detectors may be devices used to measure the flux, spatial distribution, spectrum or other properties of radiations.

Radiation detectors may be used for many applications. One important application is imaging. Radiation imaging is a radiography technique and can be used to reveal the internal structure of a non-uniformly composed and opaque object such as the human body.

Early radiation detectors for imaging include photographic plates and photographic films. A photographic plate may be a glass plate with a coating of light-sensitive emulsion. Although photographic plates were replaced by photographic films, they may still be used in special situations due to the superior quality they offer and their extreme stability. A photographic film may be a plastic film (e.g., a strip or sheet) with a coating of light-sensitive emulsion.

In the 1980s, photostimulable phosphor plates (PSP plates) became available. A PSP plate may contain a phosphor material with color centers in its lattice. When the PSP plate is exposed to radiation, electrons excited by radiation are trapped in the color centers until they are stimulated by a laser beam scanning over the plate surface. As the plate is scanned by laser, trapped excited electrons give off light, which is collected by a photomultiplier tube. The collected light is converted into a digital image. In contrast to photographic plates and photographic films, PSP plates can be reused.

Another kind of radiation detectors are radiation image intensifiers. Components of a radiation image intensifier are usually sealed in a vacuum. In contrast to photographic plates, photographic films, and PSP plates, radiation image intensifiers may produce real-time images, i.e., do not require post-exposure processing to produce images. Radiation first hits an input phosphor (e.g., cesium iodide) and is converted to visible light. The visible light then hits a photocathode (e.g., a thin metal layer containing cesium and antimony compounds) and causes emission of electrons. The number of emitted electrons is proportional to the intensity of the incident radiation. The emitted electrons are projected, through electron optics, onto an output phosphor and cause the output phosphor to produce a visible-light image.

Scintillators operate somewhat similarly to radiation image intensifiers in that scintillators (e.g., sodium iodide) absorb radiation and emit visible light, which can then be detected by a suitable image sensor for visible light. In scintillators, the visible light spreads and scatters in all directions and thus reduces spatial resolution. Reducing the scintillator thickness helps to improve the spatial resolution but also reduces absorption of radiation. A scintillator thus has to strike a compromise between absorption efficiency and resolution.

Semiconductor radiation detectors largely overcome this problem by direct conversion of radiation into electric signals. A semiconductor radiation detector may include a semiconductor layer that absorbs radiation in wavelengths of interest. When a particle of radiation is absorbed in the semiconductor layer, multiple charge carriers (e.g., electrons and holes) are generated and swept under an electric field towards electric contacts on the semiconductor layer. Cumbersome heat management required in currently available semiconductor radiation detectors (e.g., Medipix) can make a detector with a large area and a large number of pixels difficult or impossible to produce.

### SUMMARY

Disclosed herein is an image sensor comprising: a first radiation detector, a second radiation detector, and a third radiation detector, each of which comprising a planar surface configured to receive radiation from a radiation source; wherein the planar surfaces of the first radiation detector and the second radiation detector are not parallel, the planar surfaces of the second radiation detector and the third radiation detector are not parallel, and the planar surfaces of the third radiation detector and the first radiation detector are not parallel; wherein the first radiation detector, the second radiation detector and the third radiation detector are not arranged in the same row; wherein the first radiation detector, the second radiation detector and the third radiation detector are configured such that the planar surface of each of the first radiation detector, the second radiation detector and the third radiation detector includes a position at which an angle of incidence of the radiation from the radiation source is 0°.

According to an embodiment, the first radiation detector and the second radiation detector are mounted on a first support; wherein the third radiation detector is mounted on a second support.

According to an embodiment, the first radiation detector and the second radiation detector are respectively mounted on two mutually unparallel faces of the first support.

According to an embodiment, first support comprises a back face opposite to the first radiation detector and the second radiation detector; wherein the second support comprises a back face opposite to the third radiation detector.

According to an embodiment, the first support comprises a through hole extending from the back face of the first support to the first radiation detector, the through hole configured to accommodate a cable connected to the first radiation detector.

According to an embodiment, the first support is not directly joined with the second support.

According to an embodiment, the first support and the second support are mounted to a system support such that the back faces of the first support and the second support are not parallel.

According to an embodiment, the first support and the second support are mounted to two mutually unparallel faces of the system support.

According to an embodiment, the first support and the second support are spaced apart.

According to an embodiment, the first radiation detector, the second radiation detector and the third radiation detector are configured to move relative to the radiation source, to a plurality of positions; wherein the image sensor is configured to capture, by using the first radiation detector, the second radiation detector and the third radiation detector with the radiation, images of portions of a scene respectively at the positions, and configured to form an image of the scene by stitching the images of the portions.

According to an embodiment, the first radiation detector, the second radiation detector and the third radiation detector are configured to move relative to the radiation source by rotating about a first axis relative to the radiation source.

According to an embodiment, the first radiation detector, the second radiation detector and the third radiation detector are configured to move relative to the radiation source by rotating about a second axis relative to the radiation source; wherein the second axis is different from the first axis.

According to an embodiment, the radiation source is on the first axis.

According to an embodiment, the first radiation detector, the second radiation detector and the third radiation detector are configured to move relative to the radiation source by translating along a first direction relative to the radiation source.

According to an embodiment, the first direction is parallel to the planar surface of the first radiation detector and the planar surface of the second radiation detector.

According to an embodiment, the first direction is parallel to the planar surface of the first radiation detector but not parallel to the planar surface of the second radiation detector.

According to an embodiment, the first radiation detector, the second radiation detector and the third radiation detector are configured to move relative to the radiation source by translating along a second direction relative to the radiation source; wherein the second direction is different from the first direction.

According to an embodiment, the first radiation detector, the second radiation detector and the third radiation detector each comprise an array of pixels.

According to an embodiment, at least one of the first radiation detector, the second radiation detector and the third radiation detector is rectangular in shape.

According to an embodiment, at least one of the first radiation detector, the second radiation detector and the third radiation detector is hexagonal in shape.

According to an embodiment, at least one of the first radiation detector, the second radiation detector and the third radiation detector comprises a radiation absorption layer and an electronics layer; wherein the radiation absorption layer comprises an electrode; wherein the electronics layer comprises an electronics system; wherein the electronics system comprises: a first voltage comparator configured to compare a voltage of the electrode to a first threshold, a second voltage comparator configured to compare the voltage to a second threshold, a counter configured to register a number of particles of radiation reaching the radiation absorption layer, and a controller; wherein the controller is configured to start a time delay from a time at which the first voltage comparator determines that an absolute value of the voltage equals or exceeds an absolute value of the first threshold; wherein the controller is configured to activate the second voltage comparator during the time delay; wherein the controller is configured to cause the number registered by the counter to increase by one, if the second voltage comparator determines that an absolute value of the voltage equals or exceeds an absolute value of the second threshold.

According to an embodiment, the electronics system further comprises a capacitor module electrically connected to the electrode, wherein the capacitor module is configured to collect charge carriers from the electrode.

According to an embodiment, the controller is configured to activate the second voltage comparator at a beginning or expiration of the time delay.

According to an embodiment, the electronics system further comprises a voltmeter, wherein the controller is configured to cause the voltmeter to measure the voltage upon expiration of the time delay.

According to an embodiment, the controller is configured to determine energy of a particle of radiation based on a value of the voltage measured upon expiration of the time delay.

According to an embodiment, the controller is configured to connect the electrode to an electrical ground.

According to an embodiment, a rate of change of the voltage is substantially zero at expiration of the time delay.

According to an embodiment, a rate of change of the voltage is substantially non-zero at expiration of the time delay.

Disclosed herein is a system comprising the image sensor described above and a radiation source, wherein the system is configured to perform radiography on human breasts.

### BRIEF DESCRIPTION OF FIGURES

FIG. 1A schematically shows a perspective view of an image sensor comprising a plurality of radiation detectors, according to an embodiment.

FIG. 1B schematically shows a cross-sectional view of a portion of a first support of the image sensor, according to an embodiment.

FIG. 1C schematically shows a perspective view and a lateral view of a first support and a second support mounted on a system support, according to an embodiment.

FIG. 2A schematically shows a cross-sectional view of a radiation detector, according to an embodiment.

FIG. 2B schematically shows a detailed cross-sectional view of the radiation detector, according to an embodiment.

FIG. 2C schematically shows an alternative detailed cross-sectional view of the radiation detector, according to an embodiment.

FIG. 3 schematically shows that the radiation detector may have an array of pixels, according to an embodiment.

FIG. 4 schematically shows a functional block diagram of the image sensor, according to an embodiment.

FIG. 5A and FIG. 5B each schematically show movements of the image sensor relative to a radiation source, according to an embodiment.

FIG. 6 schematically shows the image sensor capturing images of portions of a scene, according to an embodiment.

FIG. 7A-7C schematically show arrangements of the radiation detectors in the image sensor, according to some embodiments.

FIG. 8 schematically shows an image sensor with plurality of radiation detectors that are hexagonal in shape, according to an embodiment.

FIG. 9 schematically shows a system comprising the image sensor described herein, suitable for medical imaging such as chest radiation radiography, abdominal radiation radiography, etc., according to an embodiment

FIG. 10 schematically shows a system comprising the image sensor described herein suitable for dental radiation radiography, according to an embodiment.

FIG. 11 schematically shows a cargo scanning or non-intrusive inspection (NII) system comprising the image sensor described herein, according to an embodiment.

FIG. 12 schematically shows another cargo scanning or non-intrusive inspection (NII) system comprising the image sensor described herein, according to an embodiment.

FIG. 13 schematically shows a full-body scanner system comprising the image sensor described herein, according to an embodiment.

FIG. 14 schematically shows a radiation computed tomography (Radiation CT) system comprising the image sensor described herein, according to an embodiment.

FIG. 15 schematically shows an electron microscope comprising the image sensor described herein, according to an embodiment.

FIG. 16A and FIG. 16B each show a component diagram of an electronic system of the radiation detector in FIG. 2A, FIG. 2B and FIG. 2C, according to an embodiment.

FIG. 17 schematically shows a temporal change of the electric current flowing through an electrode (upper curve) of a diode or an electric contact of a resistor of a radiation absorption layer exposed to radiation, the electric current caused by charge carriers generated by a particle of radiation incident on the radiation absorption layer, and a corresponding temporal change of the voltage of the electrode (lower curve), according to an embodiment.

### DETAILED DESCRIPTION

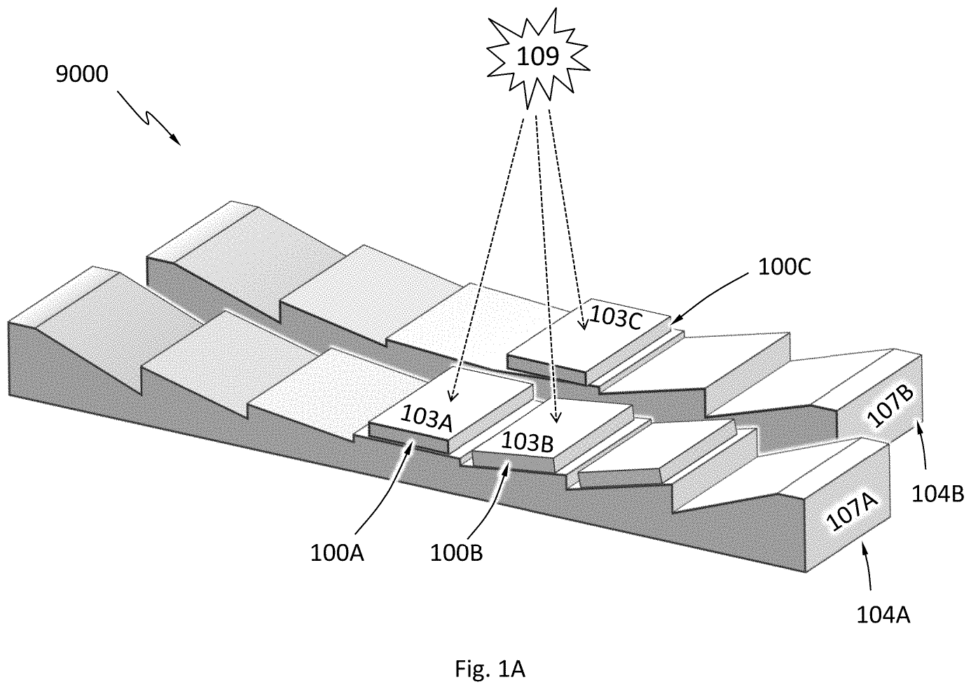

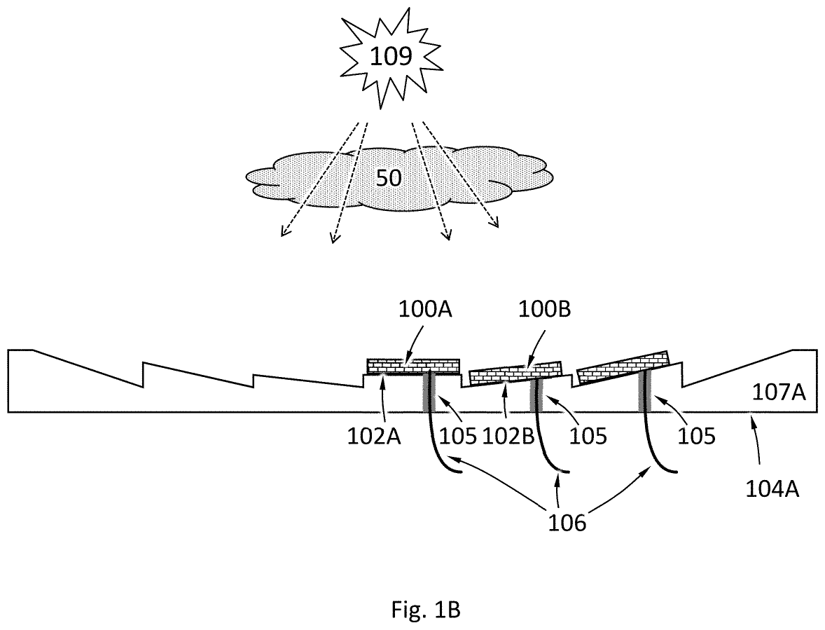

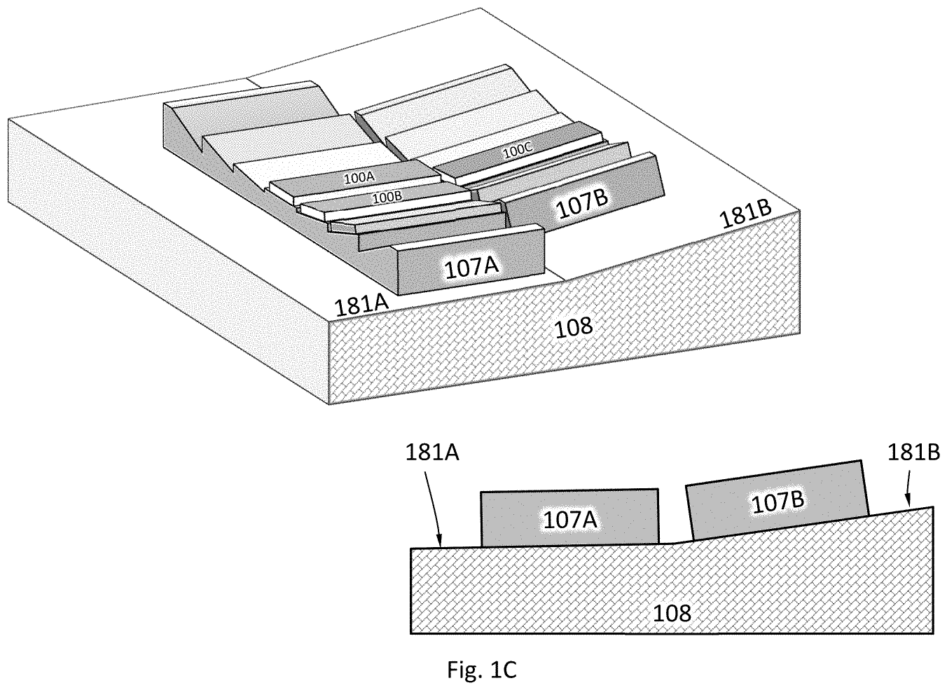

FIG. 1A schematically shows a perspective view of an image sensor 9000 comprising a plurality of radiation detectors 100 (e.g., a first radiation detector 100A, a second radiation detector 100B, a third radiation detector 100C), according to an embodiment. For brevity, only three radiation detectors are shown but the image sensor 9000 may have many more radiation detectors. Each of the radiation detectors 100 may comprise a planar surface configured to receive radiation from a radiation source 109. Namely, the first radiation detector 100A may have a planar surface 103A configured to receive radiation from the radiation source 109, the second radiation detector 100B may have a planar surface 103B configured to receive radiation from the radiation source 109, the third radiation detector 100C may have a planar surface 103C configured to receive radiation from the radiation source 109, respectively. In an embodiment, the planar surfaces (e.g. 103A and 103B) of the first radiation detector 100A and the second radiation detector 100B are not parallel, the planar surfaces (e.g. 103B and 103C) of the second radiation detector 100B and the third radiation detector 100C are not parallel, and the planar surfaces (e.g. 103C and 103A) of the third radiation detector 100C and the first radiation detector 100A are not parallel. The planar surface 103A of the first radiation detector 100A is arranged such that it may have a position at which an angle of incidence of the radiation from the radiation source 109 is 0°. The planar surface 103B of the second radiation detector 100B is arranged such that it may have a position at which an angle of incidence of the radiation from the radiation source 109 is 0°. The planar surface 103C of the third radiation detector 100C is arranged such that it may have a position at which an angle of incidence of the radiation from the radiation source 109 is 0°.

The plurality of radiation detectors 100 are arranged on a plurality of supports 107 (e.g. a first support 107A, a second support 107B), according to an embodiment. FIG. 1A shows that the first radiation detector 100A and the second radiation detector 100B are mounted on the first support 107A, and that the third radiation detector 100C is mounted on the second support 107B. In the example of FIG. 1A, the first radiation detector 100A, the second radiation detector 100B, and the third radiation detector 100C are not arranged in the same row.

The first support 107A may comprise a back face 104A which is opposite to the first radiation detector 100A and the second radiation detector 100B. The second support 107B may comprise a back face 104B which is opposite to the third radiation detector 100C.

FIG. 1B schematically shows a cross-sectional view of a portion of the first support 107A, according to an embodiment. The first support 107A may include multiple mutually unparallel faces (e.g., 102A, 102B). The first radiation detector 100A may be mounted on a first surface 102A of the first support 107A, and the second radiation detector may be mounted on a second surface 102B of the second support 107B. The first surface 102A and the second surface 102B are mutually unparallel. The radiation from the radiation source 109 may have passed through a scene 50 (e.g., a portion of a human body) before reaching the first radiation detector 100A or the second radiation detector 100B.

According to an embodiment, the first support 107A comprises through holes 105 extending from the back face 104A of the first support 107A to the first radiation detectors, as shown in FIG. 1B. For example, one of the through hole 105 may be located near the first radiation detector 100A and configured to accommodate a cable 106 connected to the first radiation detector 100A. The other end of the cable 106 may be connected to a power supply or an electronic system for the first radiation detector 100A.

The first support 107A and the second support 107B may not be directly joined together, according to an embodiment. The first support 107A and the second support 107B may be mounted to a system support 108, as the example schematically shown in FIG. 1C. The system support 108 may include multiple mutually unparallel faces (e.g., 181A, 181B). The first support 107A is mounted to a first face 181A of the system support 108, the second support 107B is mounted to a second face 181B, such that the first support 107A and the second support 107B are spaced apart on the system support 108, and the back faces (e.g., 104A and 104B) of the first support 107A and the second support 107B are not parallel, as the example shown in the perspective view and the lateral view in FIG. 1C.

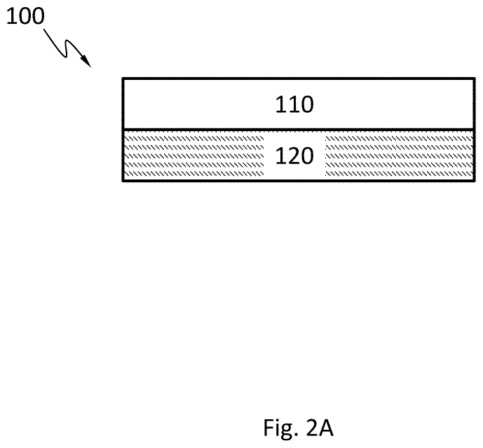

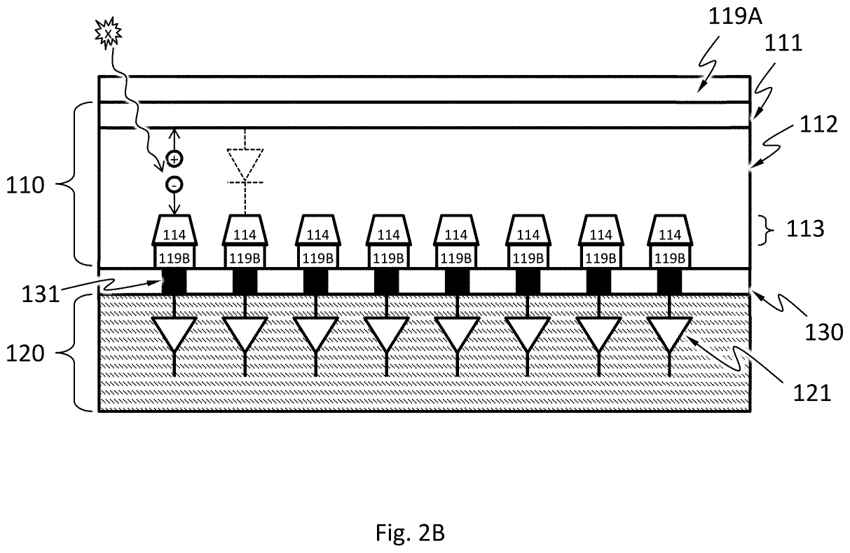

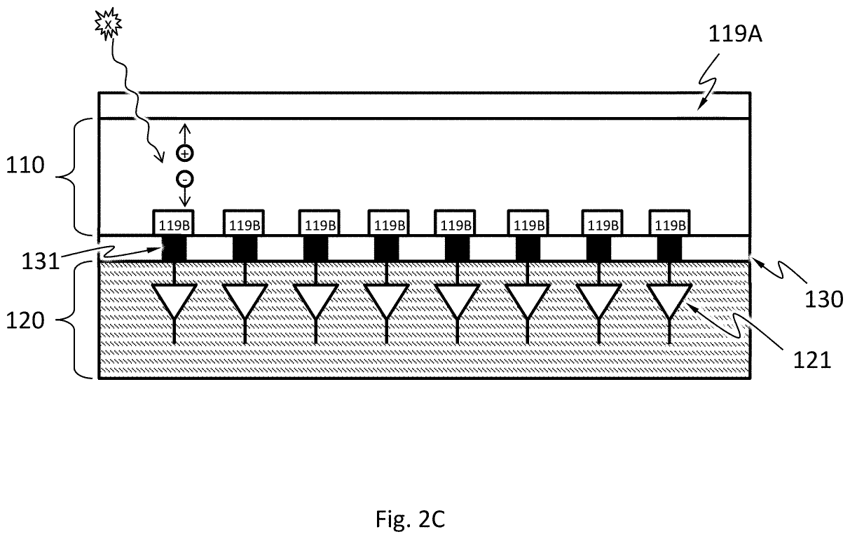

FIG. 2A schematically shows a cross-sectional view of a radiation detector 100, according to an embodiment. The radiation detector 100 may be used in the image sensor 9000. The radiation detector 100 may include a radiation absorption layer 110 and an electronics layer 120 (e.g., an ASIC) for processing or analyzing electrical signals incident radiation generates in the radiation absorption layer 110. In an embodiment, the radiation detector 100 does not comprise a scintillator. The radiation absorption layer 110 may include a semiconductor material such as, silicon, germanium, GaAs, CdTe, CdZnTe, or a combination thereof. The semiconductor may have a high mass attenuation coefficient for the radiation energy of interest. The surface of the radiation absorption layer 110 distal from the electronics layer 120 is configured to receive radiation.

As shown in a detailed cross-sectional view of the radiation detector 100 in FIG. 2B, according to an embodiment, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional the intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example in FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes having the first doped region 111 as a shared electrode. The first doped region 111 may also have discrete portions.

When a particle of radiation hits the radiation absorption layer 110 including diodes, the particle of radiation may be absorbed and generate one or more charge carriers by a number of mechanisms. A particle of radiation may generate 10 to 100000 charge carriers. The charge carriers may drift to the electrodes of one of the diodes under an electric field. The field may be an external electric field. The electric contact 119B may include discrete portions each of which is in electrical contact with the discrete regions 114. In an embodiment, the charge carriers may drift in directions such that the charge carriers generated by a single particle of radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of radiation incident around the footprint of one of these discrete regions 114 are not substantially shared with another of these discrete regions 114. A pixel 150 associated with a discrete region 114 may be an area around the discrete region 114 in which substantially all (more than 98%, more than 99.5%, more than 99.9%, or more than 99.99% of) charge carriers generated by a particle of radiation incident therein at an angle of incidence of 0° flow to the discrete region 114. Namely, less than 2%, less than 1%, less than 0.1%, or less than 0.01% of these charge carriers flow beyond the pixel.

As shown in an alternative detailed cross-sectional view of the radiation detector 100 in FIG. 2C, according to an embodiment, the radiation absorption layer 110 may include a resistor of a semiconductor material such as, silicon, germanium, GaAs, CdTe, CdZnTe, or a combination thereof, but does not include a diode. The semiconductor may have a high mass attenuation coefficient for the radiation energy of interest.

When a particle of radiation hits the radiation absorption layer 110 including a resistor but not diodes, it may be absorbed and generate one or more charge carriers by a number of mechanisms. A particle of radiation may generate 10 to 100000 charge carriers. The charge carriers may drift to the electric contacts 119A and 119B under an electric field. The field may be an external electric field. The electric contact 119B includes discrete portions. In an embodiment, the charge carriers may drift in directions such that the charge carriers generated by a single particle of radiation are not substantially shared by two different discrete portions of the electric contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of radiation incident around the footprint of one of these discrete portions of the electric contact 119B are not substantially shared with another of these discrete portions of the electric contact 119B. A pixel 150 associated with a discrete portion of the electric contact 119B may be an area around the discrete portion in which substantially all (more than 98%, more than 99.5%, more than 99.9% or more than 99.99% of) charge carriers generated by a particle of radiation incident at an angle of incidence of 0° therein flow to the discrete portion of the electric contact 119B. Namely, less than 2%, less than 0.5%, less than 0.1%, or less than 0.01% of these charge carriers flow beyond the pixel associated with the one discrete portion of the electric contact 119B.

The electronics layer 120 may include an electronic system 121 suitable for processing or interpreting signals generated by particles of radiation incident on the radiation absorption layer 110. The electronic system 121 may include an analog circuitry such as a filter network, amplifiers, integrators, and comparators, or a digital circuitry such as a microprocessor, and memory. The electronic system 121 may include components shared by the pixels or components dedicated to a single pixel. For example, the electronic system 121 may include an amplifier dedicated to each pixel and a microprocessor shared among all the pixels. The electronic system 121 may be electrically connected to the pixels by vias 131. Space among the vias may be filled with a filler material 130, which may increase the mechanical stability of the connection of the electronics layer 120 to the radiation absorption layer 110. Other bonding techniques are possible to connect the electronic system 121 to the pixels without using vias.

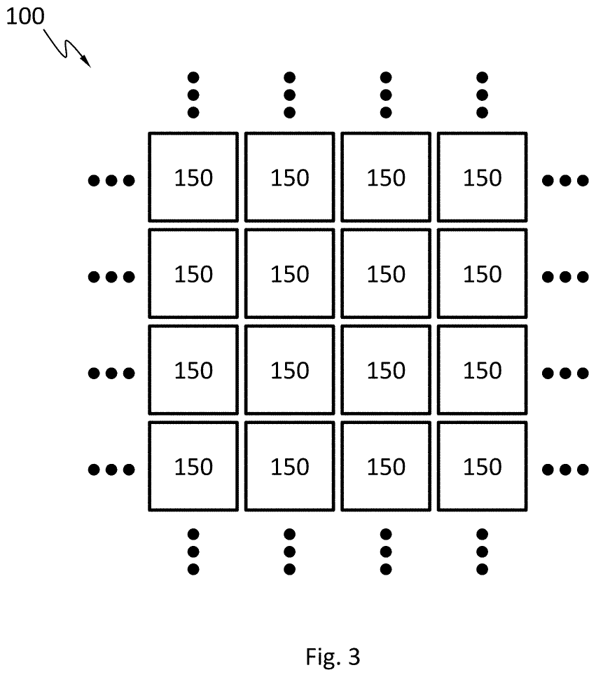

FIG. 3 schematically shows that the radiation detector 100 (e.g., the first radiation detector 100A, the second radiation detector 1008, and the third radiation detector 100C) may each have an array of pixels 150. The array may be a rectangular array, a honeycomb array, a hexagonal array or any other suitable array. Each pixel 150 may be configured to detect a particle of radiation incident thereon, to measure the energy of the particle of radiation, or both. For example, each pixel 150 may be configured to count numbers of particles of radiation incident thereon whose energy falls in a plurality of bins, within a period of time. All the pixels 150 may be configured to count the numbers of particles of radiation incident thereon within a plurality of bins of energy within the same period of time. Each pixel 150 may have its own analog-to-digital converter (ADC) configured to digitize an analog signal representing the energy of an incident particle of radiation into a digital signal. The ADC may have a resolution of 10 bits or higher. Each pixel 150 may be configured to measure its dark current, such as before or concurrently with each particle of radiation incident thereon. Each pixel 150 may be configured to deduct the contribution of the dark current from the energy of the particle of radiation incident thereon. The pixels 150 may be configured to operate in parallel. For example, when one pixel 150 measures an incident particle of radiation, another pixel 150 may be waiting for another particle of radiation to arrive. The pixels 150 may be but do not have to be individually addressable.

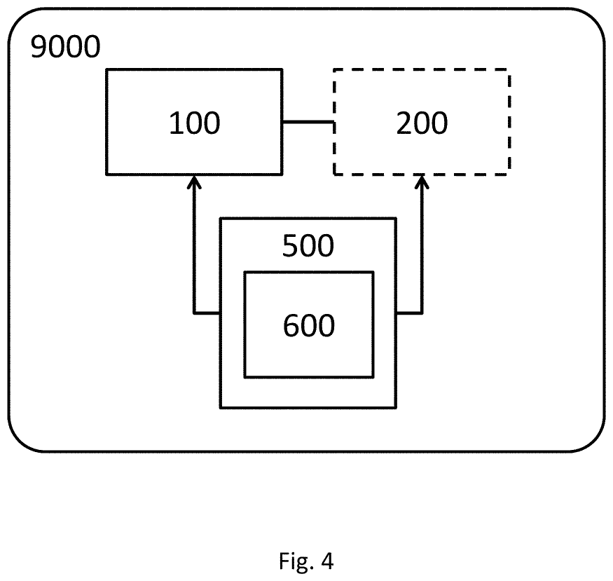

In an embodiment, the radiation detectors 100 (e.g., 100A, 1008, and 100C) of the image sensor 9000 can move to a plurality of positions, relative to the radiation source 109. The image sensor 9000 may use the radiation detectors 100 and with the radiation from the radiation source 109 to capture images of multiple portions of the scene 50 respectively at the multiple positions. The image sensor 9000 can stitch these images to form an image of the entire scene 50. As shown in FIG. 4, according to an embodiment, the image sensor 9000 may include an actuator 500 configured to move the radiation detectors 100 to the multiple positions. The actuator 500 may include a controller 600. The image sensor may include a collimator 200 that only allows radiation to reach active area of the radiation detectors 100. Active areas of the radiation detectors 100 are areas of the radiation detectors 100 that are sensitive to the radiation. The actuator 500 may move the collimator 200 together with the radiation detectors 100. The positions may be determined by the controller 600.

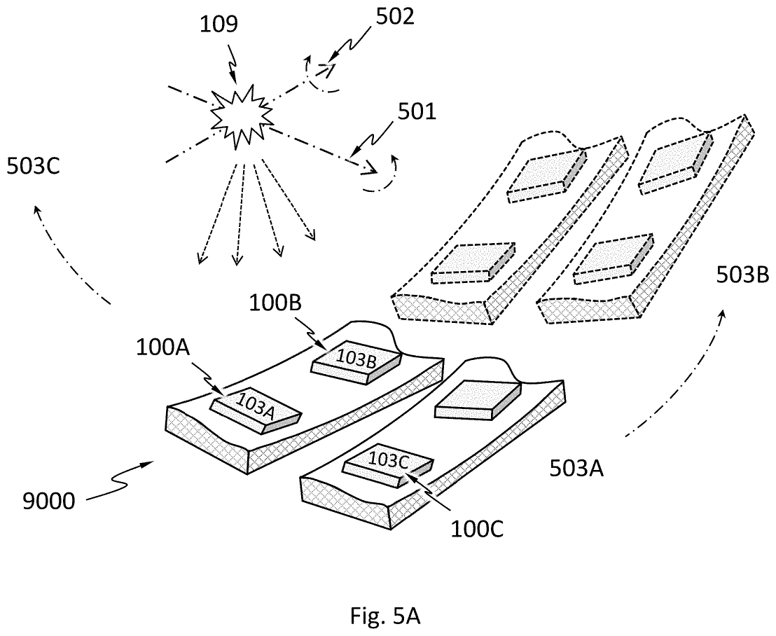

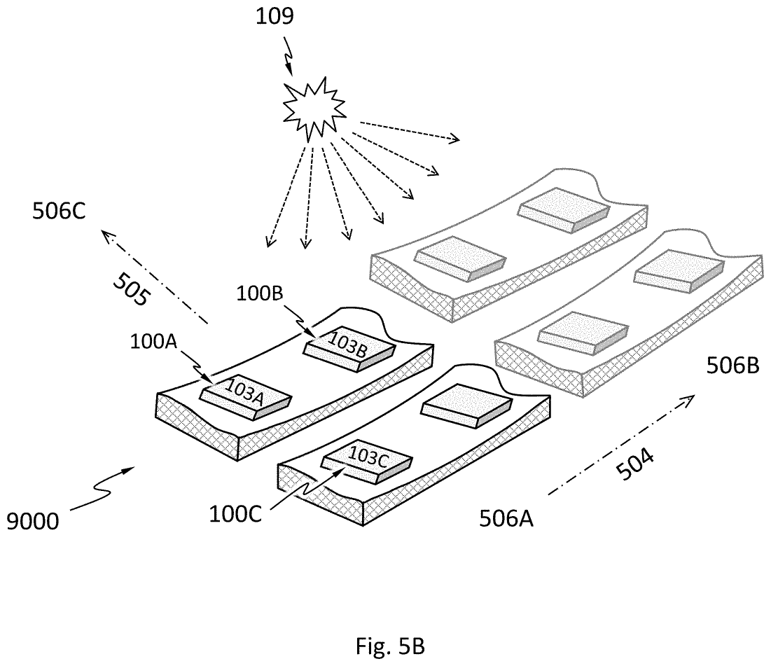

FIG. 5A and FIG. 5B each schematically shows movements of the radiation detectors 100 (e.g., 100A, 100B, 100C) relative to the radiation source 109, according to an embodiment. In the examples of FIG. 5A and FIG. 5B, only a portion of the image sensor 9000 with the first radiation detector 100A, the second radiation detector 100B and the third radiation detector 100C are shown. Relative positions of the first radiation detector 100A with respect to the second radiation detector 100B and the third radiation detector 100C remain the same at the multiple positions. The first radiation detector 100A, the second radiation detector 100B, and the third radiation detector 100C may rotate about a first axis 501, relative to the radiation source 109. As shown in the example of FIG. 5A, the first radiation detector 100A, the second radiation detector 100B and the third radiation detector 100C rotate from position 503A to position 503B about the first axis 501, relative to the radiation source 109. The first axis 501 may be parallel to the first planar surface 103A of the first radiation detector 100A and the second planar surface 103B of the second radiation detector 100B. The radiation source may be on the first axis 501. The first radiation detector 100A, the second radiation detector 100B and the third radiation detector 100C may rotate about a second axis 502 relative to the radiation source 109. The second axis 502 is different from the first axis 501. For example, the second axis 502 may be perpendicular to the first axis 501. As shown in the example of FIG. 5A, the first radiation detector 100A, the second radiation detector 100B and the third radiation detector 100C can rotate from position 503A to position 503C, about the second axis 502. The radiation source 109 may be on the second axis 502.

As shown in the example of FIG. 5B, the first radiation detector 100A, the second radiation detector 100B and the third radiation detector 100C translate along a first direction 504 from position 506A to position 506B, relative to the radiation source 109. The first radiation detector 100A, the second radiation detector 100B and the third radiation detector 100C may translate along a second direction 505. The second direction 505 is different from the first direction 504. For example, the second direction 505 may be perpendicular to the first direction 504. As shown in the example of FIG. 5B, the first radiation detector 100A, the second radiation detector 100B and the third radiation detector 100C can translated from position 506A to position 506C, along the second direction 505. The first direction 504 or the second direction 505 may be parallel to both, either or neither of the first planar surface 103A of the first radiation detector 100A and the second planar surface 103B of the second radiation detector 100B. For example, the first direction 504 may be parallel to the first planar surface 103A, but not parallel to the second planar surface 103B.

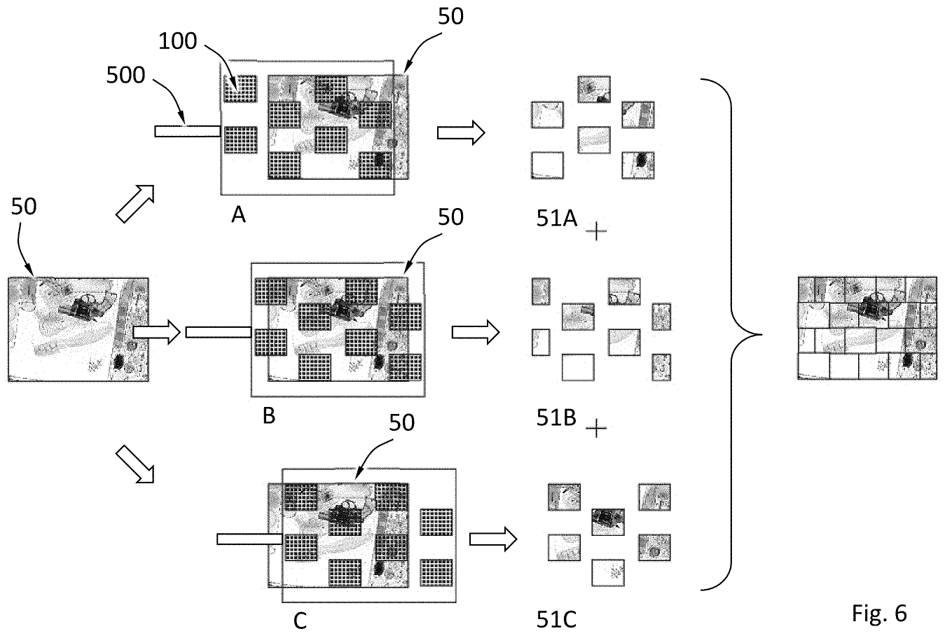

FIG. 6 schematically shows that the image sensor 9000 can capture images of portions of the scene 50 by using the first radiation detector 100A, the second radiation detector 100B and the third radiation detector 100C with the radiation, according to an embodiment. In the example shown in FIG. 6, the radiation detectors 100 move to three positions A, B and C, for example, by using the actuator 500. Respectively at the positions A, B and C, the image sensor 9000 captures images 51A, 51B and 51C of portions of the scene 50. The image sensor 9000 can stitch the images 51A, 51B and 51C of the portions to form an image of the scene 50. The images 51A, 51B and 51C of the portions may have overlap among one another to facilitate stitching. Every portion of the scene 50 may be in at least one of the images captured when the detectors are at the multiple positions. Namely, the images of the portions when stitched together may cover the entire scene 50.

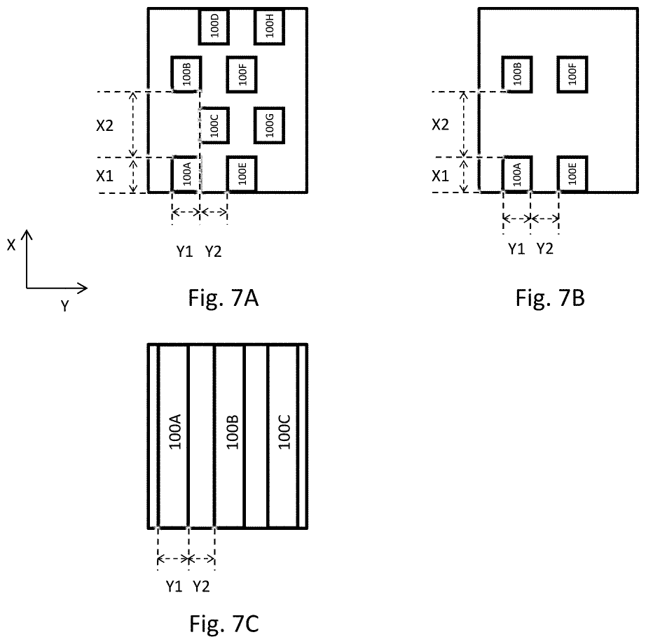

The radiation detectors 100 may be arranged in a variety of ways in the image sensor 9000. FIG. 7A schematically shows one arrangement, according to an embodiment, where the radiation detectors 100 are arranged in staggered rows. For example, radiation detectors 100A and 100B are in the same row, aligned in the Y direction, and uniform in size; radiation detectors 100C and 100D are in the same row, aligned in the Y direction, and uniform in size. Radiation detectors 100A and 100B are staggered in the X direction with respect to radiation detectors 100C and 100D. According to an embodiment, a distance X2 between two neighboring radiation detectors 100A and 100B in the same row is greater than a width X1 (i.e., dimension in the X direction, which is the extending direction of the row) of one radiation detector in the same row and is less than twice the width X1. Radiation detectors 100A and 100E are in a same column, aligned in the X direction, and uniform in size; a distance Y2 between two neighboring radiation detectors 100A and 100E in the same column is less than a width Y1 (i.e., dimension in the Y direction) of one radiation detector in the same column. This arrangement allows imaging of the scene as shown in FIG. 6, and an image of the scene may be obtained from stitching three images of portions of the scene captured at three positions spaced apart in the X direction.

FIG. 7B schematically shows another arrangement, according to an embodiment, where the radiation detectors 100 are arranged in a rectangular grid. For example, the radiation detectors 100 may include radiation detectors 100A, 100B, 100E and 100F as arranged exactly in FIG. 7A, without radiation detectors 100C, 100D, 100G, or 100H in FIG. 7A. This arrangement allows imaging of the scene by taking images of portions of the scene at six positions. For example, three positions spaced apart in the X direction and another three positions spaced apart in the X direction and spaced apart in the Y direction from the first three positions.

Other arrangements may also be possible. For example, in FIG. 7C, the radiation detectors 100 may span the whole width of the image sensor 9000 in the X-direction, with a distance Y2 between two neighboring radiation detectors 100 being less than a width of one radiation detector Y1. Assuming the width of the detectors in the X direction is greater than the width of the scene in the X direction, the image of the scene may be stitched from two images of portions of the scene captured at two positions spaced apart in the Y direction.

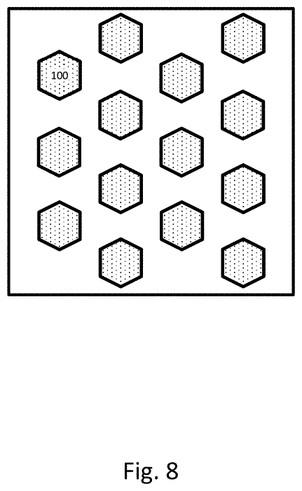

The radiation detectors 100 described above may be provided with any suitable size and shapes. According to an embodiment (e.g., in FIG. 6), at least some of the radiation detectors are rectangular in shape. According to an embodiment, as shown in FIG. 8, at least some of the radiation detectors are hexagonal in shape.

The image sensor 9000 described above may be used in various systems such as those provided below.

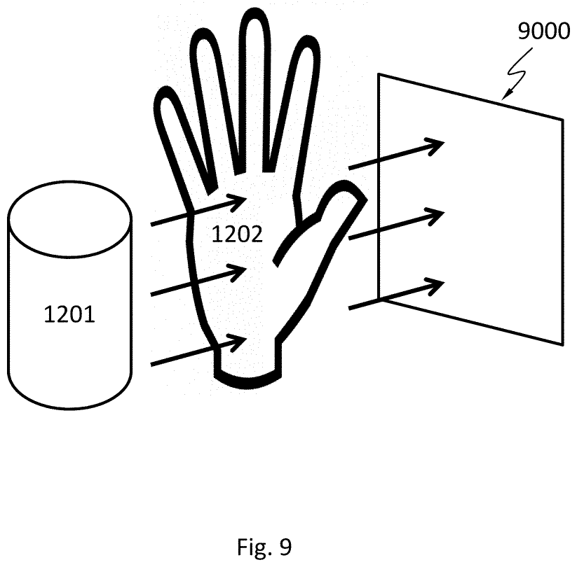

FIG. 9 schematically shows a system comprising the image sensor 9000 as described in relation to FIG. 1-FIG. 8. The system may be used for medical imaging such as chest radiation radiography, abdominal radiation radiography, etc. The system comprises a radiation source 1201. Radiation emitted from the radiation source 1201 penetrates an object 1202 (e.g., a human body part such as chest, limb, abdomen), is attenuated by different degrees by the internal structures of the object 1202 (e.g., bones, muscle, fat and organs, etc.), and is projected to the image sensor 9000. The image sensor 9000 forms an image by detecting the intensity distribution of the radiation.

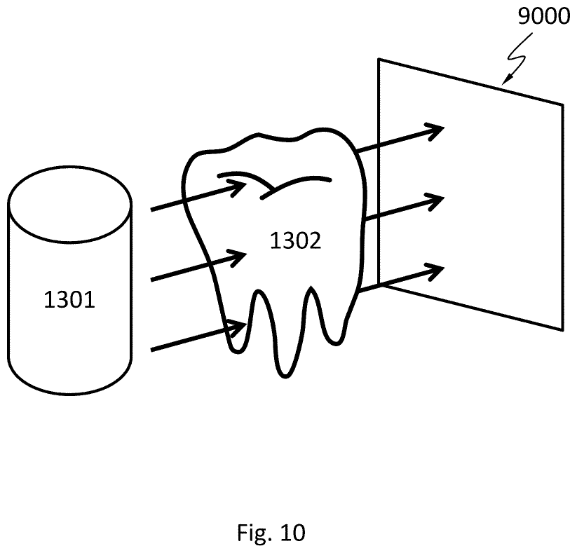

FIG. 10 schematically shows a system comprising the image sensor 9000 as described in relation to FIG. 1-FIG. 8. The system may be used for medical imaging such as dental radiation radiography. The system comprises a radiation source 1301. Radiation emitted from the radiation source 1301 penetrates an object 1302 that is part of a mammal (e.g., human) mouth. The object 1302 may include a maxilla bone, a palate bone, a tooth, the mandible, or the tongue. The radiation is attenuated by different degrees by the different structures of the object 1302 and is projected to the image sensor 9000. The image sensor 9000 forms an image by detecting the intensity distribution of the radiation. Teeth absorb radiation more than dental caries, infections, periodontal ligament. The dosage of radiation received by a dental patient is typically small (around 0.150 mSv for a full mouth series).

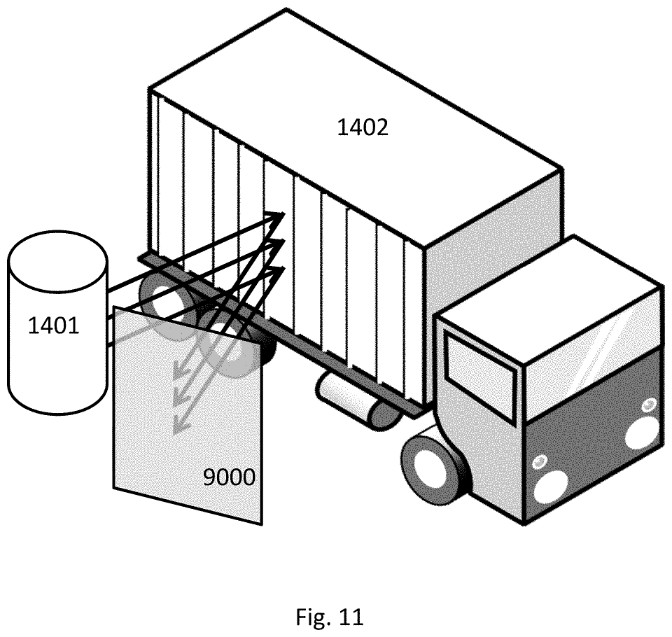

FIG. 11 schematically shows a cargo scanning or non-intrusive inspection (NII) system comprising the image sensor 9000 as described in relation to FIG. 1-FIG. 8. The system may be used for inspecting and identifying goods in transportation systems such as shipping containers, vehicles, ships, luggage, etc. The system comprises a radiation source 1401. Radiation emitted from the radiation source 1401 may backscatter from an object 1402 (e.g., shipping containers, vehicles, ships, etc.) and be projected to the image sensor 9000. Different internal structures of the object 1402 may backscatter radiation differently. The image sensor 9000 forms an image by detecting the intensity distribution of the backscattered radiation and/or energies of the backscattered particles of radiation.

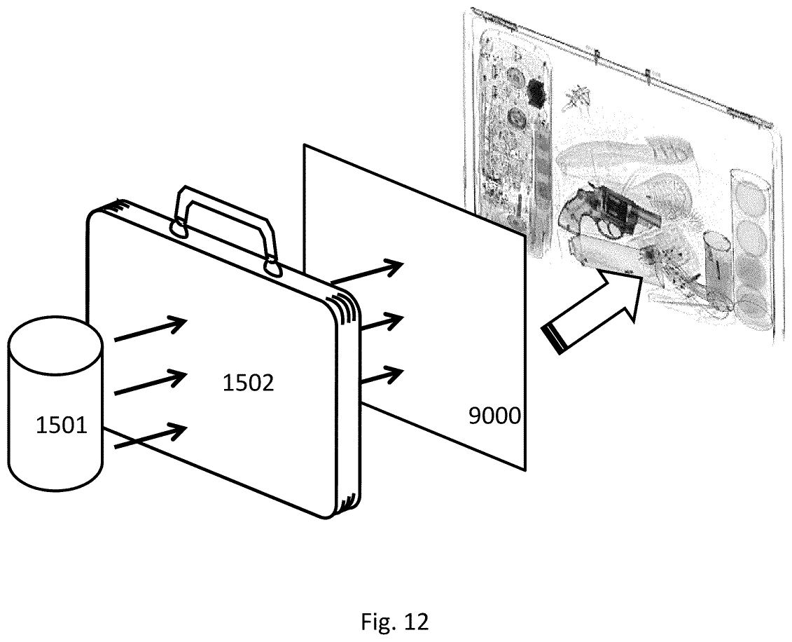

FIG. 12 schematically shows another cargo scanning or non-intrusive inspection (NII) system comprising the image sensor 9000 as described in relation to FIG. 1-FIG. 8. The system may be used for luggage screening at public transportation stations and airports. The system comprises a radiation source 1501. Radiation emitted from the radiation source 1501 may penetrate a piece of luggage 1502, be differently attenuated by the contents of the luggage, and projected to the image sensor 9000. The image sensor 9000 forms an image by detecting the intensity distribution of the transmitted radiation. The system may reveal contents of luggage and identify items forbidden on public transportation, such as firearms, narcotics, edged weapons, flammables.

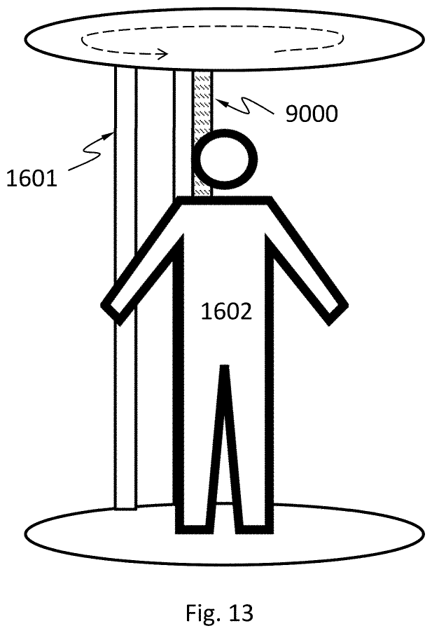

FIG. 13 schematically shows a full-body scanner system comprising the image sensor 9000 as described in relation to FIG. 1-FIG. 8. The full-body scanner system may detect objects on a person's body for security screening purposes, without physically removing clothes or making physical contact. The full-body scanner system may be able to detect non-metal objects. The full-body scanner system comprises a radiation source 1601. Radiation emitted from the radiation source 1601 may backscatter from a human 1602 being screened and objects thereon, and be projected to the image sensor 9000. The objects and the human body may backscatter radiation differently. The image sensor 9000 forms an image by detecting the intensity distribution of the backscattered radiation. The image sensor 9000 and the radiation source 1601 may be configured to scan the human in a linear or rotational direction.

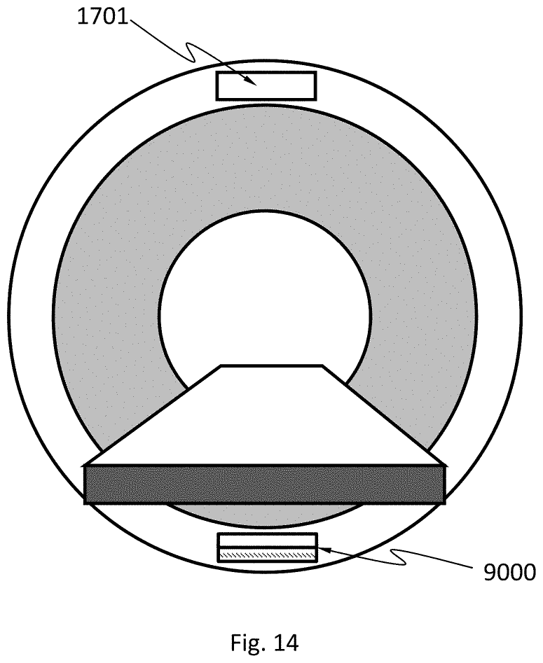

FIG. 14 schematically shows a radiation computed tomography (Radiation CT) system. The Radiation CT system uses computer-processed radiations to produce tomographic images (virtual “slices”) of specific areas of a scanned object. The tomographic images may be used for diagnostic and therapeutic purposes in various medical disciplines, or for flaw detection, failure analysis, metrology, assembly analysis and reverse engineering. The Radiation CT system comprises the image sensor 9000 as described in relation to FIG. 1-FIG. 8 and a radiation source 1701. The image sensor 9000 and the radiation source 1701 may be configured to rotate synchronously along one or more circular or spiral paths.

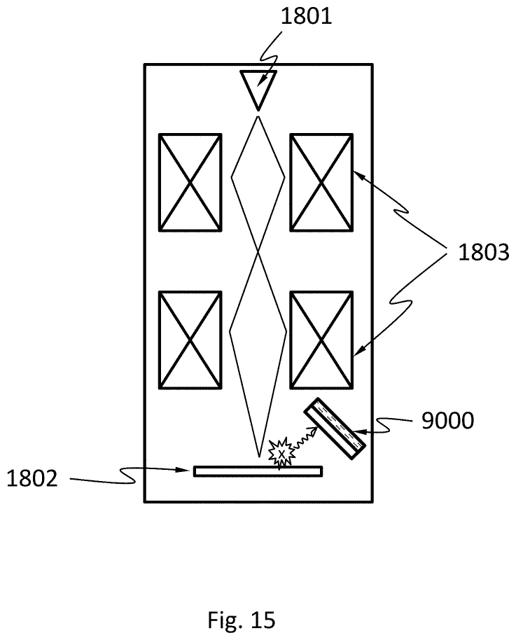

FIG. 15 schematically shows an electron microscope. The electron microscope comprises an electron source 1801 (also called an electron gun) that is configured to emit electrons. The electron source 1801 may have various emission mechanisms such as thermionic, photocathode, cold emission, or plasmas source. The emitted electrons pass through an electronic optical system 1803, which may be configured to shape, accelerate, or focus the electrons. The electrons then reach a sample 1802 and an image detector may form an image therefrom. The electron microscope may comprise the image sensor 9000 as described in relation to FIG. 1-FIG. 8, for performing energy-dispersive radiation spectroscopy (EDS). EDS is an analytical technique used for the elemental analysis or chemical characterization of a sample. When the electrons incident on a sample, they cause emission of characteristic radiations from the sample. The incident electrons may excite an electron in an inner shell of an atom in the sample, ejecting it from the shell while creating an electron hole where the electron was. An electron from an outer, higher-energy shell then fills the hole, and the difference in energy between the higher-energy shell and the lower energy shell may be released in the form of a radiation. The number and energy of the radiations emitted from the sample can be measured by the image sensor 9000.

The image sensor 9000 described here may have other applications such as in a radiation telescope, radiation mammography, industrial radiation defect detection, radiation microscopy or microradiography, radiation casting inspection, radiation non-destructive testing, radiation weld inspection, radiation digital subtraction angiography, etc. It may be suitable to use the image sensor 9000 in place of a photographic plate, a photographic film, a PSP plate, a radiation image intensifier, a scintillator, or another semiconductor radiation detector.

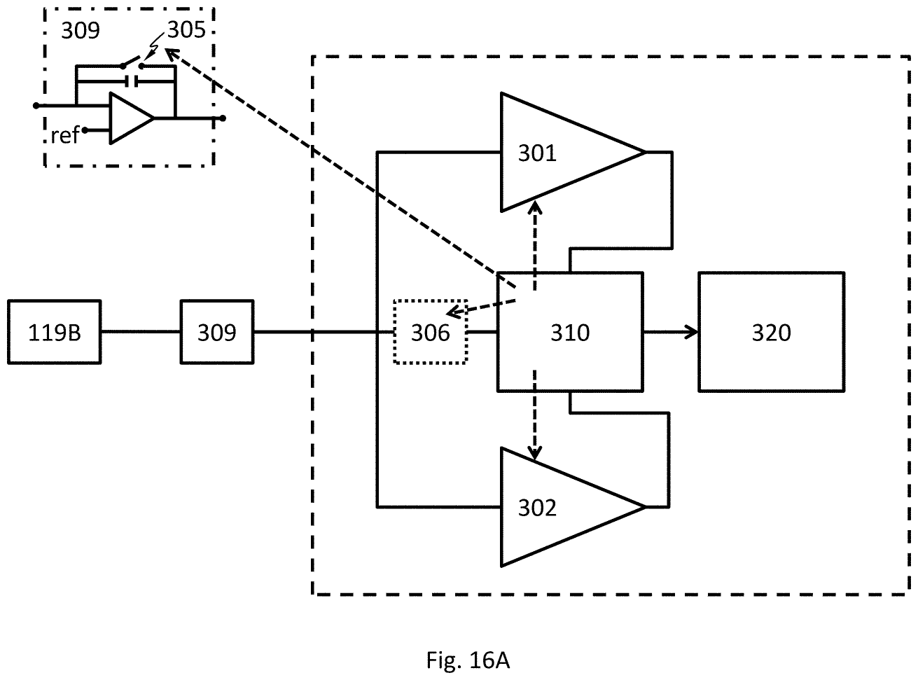

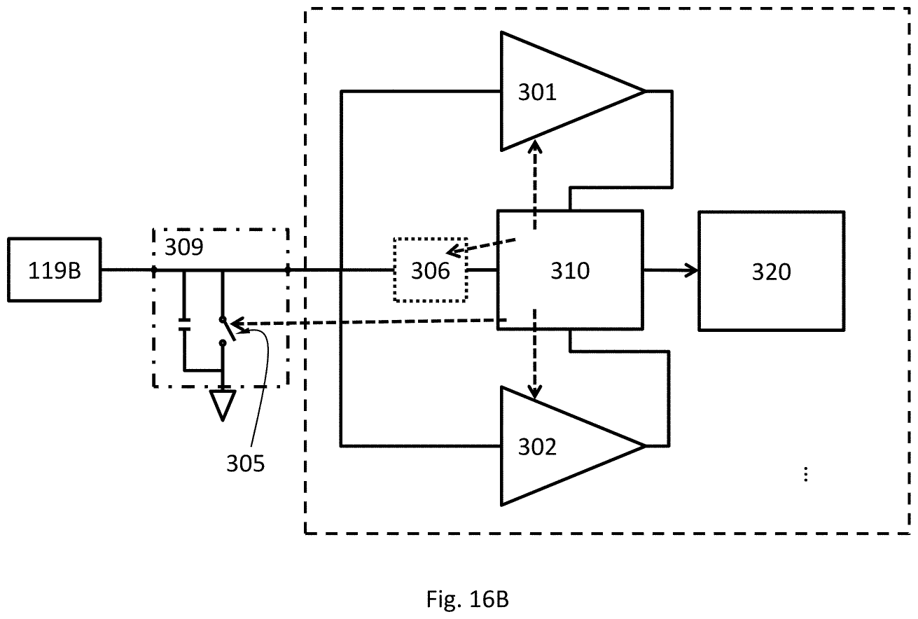

FIG. 16A and FIG. 16B each show a component diagram of the electronic system 121, according to an embodiment. The electronic system 121 may include a first voltage comparator 301, a second voltage comparator 302, a counter 320, a switch 305, an optional voltmeter 306 and a controller 310.

The first voltage comparator 301 is configured to compare the voltage of at least one of the electric contacts 119B to a first threshold. The first voltage comparator 301 may be configured to monitor the voltage directly, or calculate the voltage by integrating an electric current flowing through the electrical contact 119B over a period of time. The first voltage comparator 301 may be controllably activated or deactivated by the controller 310. The first voltage comparator 301 may be a continuous comparator. Namely, the first voltage comparator 301 may be configured to be activated continuously and monitor the voltage continuously. The first voltage comparator 301 may be a clocked comparator. The first threshold may be 5-10%, 10%-20%, 20-30%, 30-40% or 40-50% of the maximum voltage one incident particle of radiation may generate on the electric contact 119B. The maximum voltage may depend on the energy of the incident particle of radiation, the material of the radiation absorption layer 110, and other factors. For example, the first threshold may be 50 mV, 100 mV, 150 mV, or 200 mV.

The second voltage comparator 302 is configured to compare the voltage to a second threshold. The second voltage comparator 302 may be configured to monitor the voltage directly or calculate the voltage by integrating an electric current flowing through the diode or the electrical contact over a period of time. The second voltage comparator 302 may be a continuous comparator. The second voltage comparator 302 may be controllably activate or deactivated by the controller 310. When the second voltage comparator 302 is deactivated, the power consumption of the second voltage comparator 302 may be less than 1%, less than 5%, less than 10% or less than 20% of the power consumption when the second voltage comparator 302 is activated. The absolute value of the second threshold is greater than the absolute value of the first threshold. As used herein, the term “absolute value” or “modulus” |x| of a real number x is the non-negative value of x without regard to its sign. Namely,

$$\vert x \vert = \{ \begin{matrix}x , if x \geq 0 \\ - x , if x \leq 0\end{matrix} .$$

The second threshold may be 200%-300% of the first threshold. The second threshold may be at least 50% of the maximum voltage one incident particle of radiation may generate on the electric contact 119B. For example, the second threshold may be 100 mV, 150 mV, 200 mV, 250 mV or 300 mV. The second voltage comparator 302 and the first voltage comparator 310 may be the same component. Namely, the system 121 may have one voltage comparator that can compare a voltage with two different thresholds at different times.

The first voltage comparator 301 or the second voltage comparator 302 may include one or more op-amps or any other suitable circuitry. The first voltage comparator 301 or the second voltage comparator 302 may have a high speed to allow the electronic system 121 to operate under a high flux of incident particles of radiation. However, having a high speed is often at the cost of power consumption.

The counter 320 is configured to register at least a number of particles of radiation incident on the pixel 150 encompassing the electric contact 119B. The counter 320 may be a software component (e.g., a number stored in a computer memory) or a hardware component (e.g., a 4017 IC and a 7490 IC).

The controller 310 may be a hardware component such as a microcontroller and a microprocessor. The controller 310 is configured to start a time delay from a time at which the first voltage comparator 301 determines that the absolute value of the voltage equals or exceeds the absolute value of the first threshold (e.g., the absolute value of the voltage increases from below the absolute value of the first threshold to a value equal to or above the absolute value of the first threshold). The absolute value is used here because the voltage may be negative or positive, depending on whether the voltage of the cathode or the anode of the diode or which electrical contact is used. The controller 310 may be configured to keep deactivated the second voltage comparator 302, the counter 320 and any other circuits the operation of the first voltage comparator 301 does not require, before the time at which the first voltage comparator 301 determines that the absolute value of the voltage equals or exceeds the absolute value of the first threshold. The time delay may expire before or after the voltage becomes stable, i.e., the rate of change of the voltage is substantially zero. The phase “the rate of change of the voltage is substantially zero” means that temporal change of the voltage is less than 0.1%/ns. The phase “the rate of change of the voltage is substantially non-zero” means that temporal change of the voltage is at least 0.1%/ns.

The controller 310 may be configured to activate the second voltage comparator during (including the beginning and the expiration) the time delay. In an embodiment, the controller 310 is configured to activate the second voltage comparator at the beginning of the time delay. The term “activate” means causing the component to enter an operational state (e.g., by sending a signal such as a voltage pulse or a logic level, by providing power, etc.). The term “deactivate” means causing the component to enter a non-operational state (e.g., by sending a signal such as a voltage pulse or a logic level, by cut off power, etc.). The operational state may have higher power consumption (e.g., 10 times higher, 100 times higher, 1000 times higher) than the non-operational state. The controller 310 itself may be deactivated until the output of the first voltage comparator 301 activates the controller 310 when the absolute value of the voltage equals or exceeds the absolute value of the first threshold.

The controller 310 may be configured to cause at least one of the number registered by the counter 320 to increase by one, if, during the time delay, the second voltage comparator 302 determines that the absolute value of the voltage equals or exceeds the absolute value of the second threshold.

The controller 310 may be configured to cause the optional voltmeter 306 to measure the voltage upon expiration of the time delay. The controller 310 may be configured to connect the electric contact 119B to an electrical ground, so as to reset the voltage and discharge any charge carriers accumulated on the electric contact 119B. In an embodiment, the electric contact 119B is connected to an electrical ground after the expiration of the time delay. In an embodiment, the electric contact 119B is connected to an electrical ground for a finite reset time period. The controller 310 may connect the electric contact 119B to the electrical ground by controlling the switch 305. The switch may be a transistor such as a field-effect transistor (FET).

In an embodiment, the system 121 has no analog filter network (e.g., a RC network). In an embodiment, the system 121 has no analog circuitry.

The voltmeter 306 may feed the voltage it measures to the controller 310 as an analog or digital signal.

The electronic system 121 may include an integrator 309 electrically connected to the electric contact 119B, wherein the integrator is configured to collect charge carriers from the electric contact 119B. The integrator 309 can include a capacitor in the feedback path of an amplifier. The amplifier configured as such is called a capacitive transimpedance amplifier (CTIA). CTIA has high dynamic range by keeping the amplifier from saturating and improves the signal-to-noise ratio by limiting the bandwidth in the signal path. Charge carriers from the electric contact 119B accumulate on the capacitor over a period of time (“integration period”). After the integration period has expired, the capacitor voltage is sampled and then reset by a reset switch. The integrator 309 can include a capacitor directly connected to the electric contact 119B.

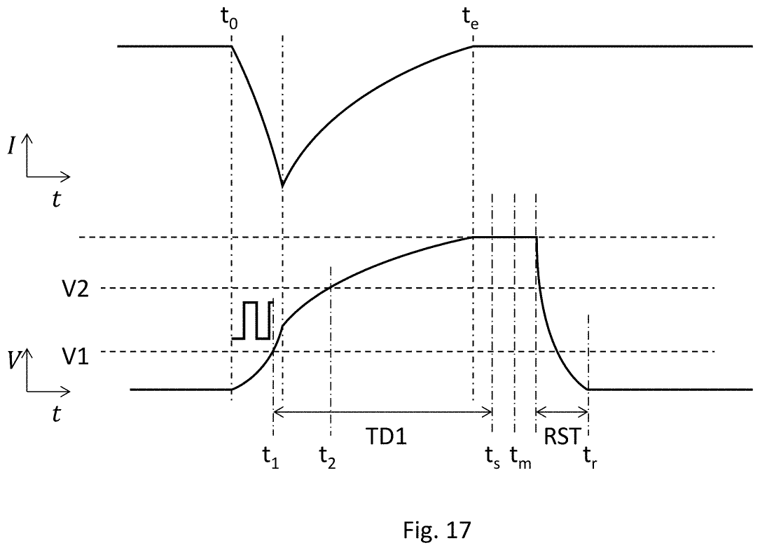

FIG. 17 schematically shows a temporal change of the electric current flowing through the electric contact 119B (upper curve) caused by charge carriers generated by a particle of radiation incident on the pixel 150 encompassing the electric contact 119B, and a corresponding temporal change of the voltage of the electric contact 119B (lower curve). The voltage may be an integral of the electric current with respect to time. At time to, the particle of radiation hits pixel 150, charge carriers start being generated in the pixel 150, electric current starts to flow through the electric contact 119B, and the absolute value of the voltage of the electric contact 119B starts to increase. At time t₁, the first voltage comparator 301 determines that the absolute value of the voltage equals or exceeds the absolute value of the first threshold V1, and the controller 310 starts the time delay TD1 and the controller 310 may deactivate the first voltage comparator 301 at the beginning of TD1. If the controller 310 is deactivated before t₁, the controller 310 is activated at t₁. During TD1, the controller 310 activates the second voltage comparator 302. The term “during” a time delay as used here means the beginning and the expiration (i.e., the end) and any time in between. For example, the controller 310 may activate the second voltage comparator 302 at the expiration of TD1. If during TD1, the second voltage comparator 302 determines that the absolute value of the voltage equals or exceeds the absolute value of the second threshold V2 at time t₂, the controller 310 waits for stabilization of the voltage to stabilize. The voltage stabilizes at time tₑ, when all charge carriers generated by the particle of radiation drift out of the radiation absorption layer 110. At time tₛ, the time delay TD1 expires. At or after time tₑ, the controller 310 causes the voltmeter 306 to digitize the voltage and determines which bin the energy of the particle of radiation falls in. The controller 310 then causes the number registered by the counter 320 corresponding to the bin to increase by one. In the example of FIG. 17, time tₛ is after time tₑ; namely TD1 expires after all charge carriers generated by the particle of radiation drift out of the radiation absorption layer 110. If time tₑ cannot be easily measured, TD1 can be empirically chosen to allow sufficient time to collect essentially all charge carriers generated by a particle of radiation but not too long to risk have another incident particle of radiation. Namely, TD1 can be empirically chosen so that time tₛ is empirically after time tₑ. Time tₛ is not necessarily after time tₑ because the controller 310 may disregard TD1 once V2 is reached and wait for time tₑ. The rate of change of the difference between the voltage and the contribution to the voltage by the dark current is thus substantially zero at tₑ. The controller 310 may be configured to deactivate the second voltage comparator 302 at expiration of TD1 or at t₂, or any time in between.

The voltage at time tₑ is proportional to the amount of charge carriers generated by the particle of radiation, which relates to the energy of the particle of radiation. The controller 310 may be configured to determine the energy of the particle of radiation, using the voltmeter 306.

After TD1 expires or digitization by the voltmeter 306, whichever later, the controller 310 connects the electric contact 119B to an electric ground for a reset period RST to allow charge carriers accumulated on the electric contact 119B to flow to the ground and reset the voltage. After RST, the electronic system 121 is ready to detect another incident particle of radiation. If the first voltage comparator 301 has been deactivated, the controller 310 can activate it at any time before RST expires. If the controller 310 has been deactivated, it may be activated before RST expires.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An image sensor comprising:
a first radiation detector, a second radiation detector, and a third radiation detector, each of which comprising a planar surface configured to receive radiation from a radiation source;
wherein the planar surfaces of the first radiation detector and the second radiation detector are not parallel, the planar surfaces of the second radiation detector and the third radiation detector are not parallel, and the planar surfaces of the third radiation detector and the first radiation detector are not parallel;
wherein the first radiation detector, the second radiation detector and the third radiation detector are not arranged in the same row;
wherein the first radiation detector, the second radiation detector and the third radiation detector are configured such that the planar surface of each of the first radiation detector, the second radiation detector and the third radiation detector includes a position at which an angle of incidence of the radiation from the radiation source is 0°;
wherein the first radiation detector, the second radiation detector and the third radiation detector are configured to move relative to the radiation source, to a plurality of positions;
wherein the image sensor is configured to capture, by using the first radiation detector, the second radiation detector and the third radiation detector with the radiation, images of portions of a scene respectively at the positions, and configured to form an image of the scene by stitching the images of the portions.

2. The image sensor of claim 1, wherein the first radiation detector and the second radiation detector are mounted on a first support; wherein the third radiation detector is mounted on a second support.

3. The image sensor of claim 2, wherein the first radiation detector and the second radiation detector are respectively mounted on two mutually unparallel faces of the first support.

4. The image sensor of claim 2, wherein the first support comprises a back face opposite to the first radiation detector and the second radiation detector; wherein the second support comprises a back face opposite to the third radiation detector.

5. The image sensor of claim 4, wherein the first support comprises a through hole extending from the back face of the first support to the first radiation detector, the through hole configured to accommodate a cable connected to the first radiation detector.

6. The image sensor of claim 2, wherein the first support is not directly joined with the second support.

7. The image sensor of claim 4, wherein the first support and the second support are mounted to a system support such that the back faces of the first support and the second support are not parallel.

8. The image sensor of claim 7, wherein the first support and the second support are mounted to two mutually unparallel faces of the system support.

9. The image sensor of claim 7, wherein the first support and the second support are spaced apart.

10. (canceled)

11. The image sensor of claim 1, wherein the first radiation detector, the second radiation detector and the third radiation detector are configured to move relative to the radiation source by rotating about a first axis relative to the radiation source.

12. The image sensor of claim 11, wherein the first radiation detector, the second radiation detector and the third radiation detector are configured to move relative to the radiation source by rotating about a second axis relative to the radiation source; wherein the second axis is different from the first axis.

13. The image sensor of claim 11, wherein the radiation source is on the first axis.

14. The image sensor of claim 1, wherein the first radiation detector, the second radiation detector and the third radiation detector are configured to move relative to the radiation source by translating along a first direction relative to the radiation source.

15. The image sensor of claim 14, wherein the first direction is parallel to the planar surface of the first radiation detector and the planar surface of the second radiation detector.

16. The image sensor of claim 14, wherein the first direction is parallel to the planar surface of the first radiation detector but not parallel to the planar surface of the second radiation detector.

17. The image sensor of claim 14, wherein the first radiation detector, the second radiation detector and the third radiation detector are configured to move relative to the radiation source by translating along a second direction relative to the radiation source; wherein the second direction is different from the first direction.

18. The image sensor of claim 1, wherein the first radiation detector, the second radiation detector and the third radiation detector each comprise an array of pixels.

19. The image sensor of claim 1, wherein at least one of the first radiation detector, the second radiation detector and the third radiation detector is rectangular in shape.

20. The image sensor of claim 1, wherein at least one of the first radiation detector, the second radiation detector and the third radiation detector is hexagonal in shape.

21. The image sensor of claim 1, wherein at least one of the first radiation detector, the second radiation detector and the third radiation detector comprises a radiation absorption layer and an electronics layer;
wherein the radiation absorption layer comprises an electrode;
wherein the electronics layer comprises an electronics system;
wherein the electronics system comprises:
a first voltage comparator configured to compare a voltage of the electrode to a first threshold,
a second voltage comparator configured to compare the voltage to a second threshold,
a counter configured to register a number of particles of radiation reaching the radiation absorption layer, and
a controller;
wherein the controller is configured to start a time delay from a time at which the first voltage comparator determines that an absolute value of the voltage equals or exceeds an absolute value of the first threshold;
wherein the controller is configured to activate the second voltage comparator during the time delay;
wherein the controller is configured to cause the number registered by the counter to increase by one, if the second voltage comparator determines that an absolute value of the voltage equals or exceeds an absolute value of the second threshold.

22. The image sensor of claim 21, wherein the electronics system further comprises a capacitor module electrically connected to the electrode, wherein the capacitor module is configured to collect charge carriers from the electrode.

23. The image sensor of claim 21, wherein the controller is configured to activate the second voltage comparator at a beginning or expiration of the time delay.

24. The image sensor of claim 21, wherein the electronics system further comprises a voltmeter, wherein the controller is configured to cause the voltmeter to measure the voltage upon expiration of the time delay.

25. The image sensor of claim 21, wherein the controller is configured to determine energy of a particle of radiation based on a value of the voltage measured upon expiration of the time delay.

26. The image sensor of claim 21, wherein the controller is configured to connect the electrode to an electrical ground.

27. The image sensor of claim 21, wherein a rate of change of the voltage is substantially zero at expiration of the time delay.

28. The image sensor of claim 21, wherein a rate of change of the voltage is substantially non-zero at expiration of the time delay.

29. A system comprising the image sensor of claim 1 and the radiation source, wherein the system is configured to perform radiography on human breasts.
